(19) United States
(12) Patent Application Publication
(10) Pub. No.: US 2026/0251811 A1
(43) Pub. Date: Aug. 27, 2026

(54) Method and System for Extracting Relational, Harmonic, and Systems-Level Features from Signals Using Multi-Order Cepstral and Spectral Analysis

(71) Applicant: Médéric Degoy, Perpignan (FR)

(72) Inventor: Médéric Degoy, Perpignan (FR)

(21) Appl. No.: 19/546,785

(22) Filed: Feb. 23, 2026

Related U.S. Application Data
(60) Provisional application No. 63/762,661, filed on Feb. 25, 2025.

(51) Int. Cl. G01V 1/30; A61B 5/00; G01S 7/35; G06V 10/62

(52) U.S. Cl. CPC G01V 1/307; A61B 5/7257; G01S 7/356; G06V 10/62

(57) ABSTRACT

A computer-implemented method extracts relational and harmonic features from time-series or multi-component signals. Cepstrum computation identifies main resonance frequencies. Common resonances across derived markers are correlated in phase space to determine link strength, treating markers as oscillators with momentum. Time-series evolution of each marker undergoes higher-order spectral analysis to derive harmonic qualities (perceptual spread, skewness, long-term trend). An integral coherence score aggregates energy, entropy, harmonic qualities, and link strengths per marker. Causal chains are constructed as navigable maps of successive resonance links, enabling energy and information flow analysis. The method applies broadly to physiological and biological measurement data, vibration monitoring, audio processing, radar/sonar, seismic data, image sequences, machine learning feature engineering, and other oscillatory or periodic signal domains.

### CROSS-REFERENCE TO RELATED APPLICATIONS

This application claims the benefit of U.S. Provisional Patent Application No. 63/762,661, filed Feb. 25, 2025, the entire contents of which are incorporated herein by reference.

### BACKGROUND OF THE INVENTION

### Field of the Invention

The present invention relates to advanced signal processing techniques for extracting relational and harmonic features from time-series or multi-component signals, applicable to fields such as physiological monitoring, audio processing, vibration analysis, radar, image processing, machine learning feature engineering, and other domains involving complex oscillatory or periodic data.

### Description of Related Art

Conventional signal processing methods often rely on first-order spectral analysis (e.g., FFT) or basic cepstral techniques for periodicity detection, noise reduction, or quality assessment. These approaches typically treat signal components independently, without deriving inter-component relationships or higher-order harmonic properties from multi-stage nonlinear transformations. There is a need for methods that extract relational links (e.g., shared resonances in phase space) and harmonic qualities (e.g., perceptual spread, asymmetry, long-term trends) from signal evolution and inter-component interactions, enabling holistic systems-level analysis across diverse applications.

### BRIEF SUMMARY OF THE INVENTION

The invention provides a multi-stage signal processing method to extract relational (link strength via common resonances in phase space) and harmonic (attention as perceptual spread, intention as skewness, horizon as long-term trend) features from raw signals. Cepstrum identifies main resonance frequencies; common resonances across markers are correlated in phase space to compute link strength (viewed as stable oscillators with momentum); beat-to-beat (or time-step) evolution of each marker undergoes higher-order spectral analysis to derive harmonic qualities; an integral coherence score aggregates energy, entropy, harmonic qualities, and link strengths per marker. Applicable broadly to signal analysis in physiological, audio, vibration, radar, image, and machine learning contexts.

### DETAILED DESCRIPTION

The invention is illustrated by way of example and not limitation in the figures of the accompanying drawings, in which like reference numerals indicate similar elements. In particular:
100 designates the Raw Signal Input
110 designates the FFT and Cepstrum Computation
120 designates the Resonance Frequencies Identification
130 designates the Markers Derivation
135 designates Energy
140 designates the Time-Series Evolution Computation
145 designates Entropy (agitation)
150 designates the Higher-Order Spectral Analysis
160 designates the Phase-Space Resonance Correlation
170 designates the Link Strength & Directed Energy Flow
180 designates the Harmonic Qualities
185 designates Attention, Intention, Horizon, cycles
190 designates the Integral Coherence Score Aggregation
200 designates the Causal Chain Map Construction
210 designates the Resources and priorities identification

The method processes a raw signal (100) (e.g., time-series data) to extract markers and relational/harmonic features (130). In a physiological setting, these markers may be referred to as biomarkers, but the technique applies generally to any signal-derived features from time-series or multi-component signals.

First stage: Compute cepstrum of the signal (110) (as a second-order transformation via log-spectrum followed by inverse FFT) to identify main resonance frequencies (120) (peaks in quefrency domain corresponding to periodic components).

Second stage: For multiple markers (130) (derived from the signal or sub-components), compute energy (135), then compute time-series evolution (140) (e.g., beat-to-beat or sample-to-sample changes) and compute entropy/agitation (145). Apply higher-order spectral analysis (150) (e.g., third-order FFT) on this evolution series to extract harmonic qualities (180)—attention (perceptual spread of the spectrum), intention (skewness/asymmetry), horizon (long-term trend or low-frequency dominance) and main cycles (185).

Third stage: Correlate common resonance frequencies across markers in phase space (160) (using phase information from FFT/cepstrum) to determine link strength (170). Links are weighted by power at shared resonances, forming “resonance bridges” between markers viewed as rotating oscillators with momentum at different rotational velocities and resonance points.

Energy transfer directionality: In linked pairs, energy flows from the marker with higher energy (source) to the one with lower energy (sink) through the common resonance, following known resonance effects that carry both information and energy (170).

Causal chains (200) are constructed as navigable 2D or higher-dimensional maps of markers connected by successive resonance links, enabling analysis of energy/information propagation paths across the signal components.

Resources and priorities identification (210): Compute an integral coherence score (IC) per marker (190) as a weighted aggregate of: energy (from cepstrum/FFT bins), entropy (agitation level), harmonic qualities (attention, intention, horizon), and relational link strengths. This score provides a holistic measure of marker stability and systemic role. Resources are markers with high IC (high energy, low agitation, balanced harmonics, strong outgoing links—“top of the food chain”). Priorities are markers with low IC (opposite characteristics—sinks or weak nodes).

The extracted features support downstream applications: relational mapping for system dynamics, predictive modeling, physiological biomarkers computation, adaptive outputs (e.g., therapeutic protocols in physiological contexts), anomaly detection, or general signal analysis in audio, vibration, radar, image processing, machine learning feature engineering, and other oscillatory/time-series domains, including but not limited to: vibration monitoring in machinery (detecting hidden fault propagation), audio processing (harmonic relationships in polyphonic sound), radar/sonar (resonant object classification), seismic data analysis (wave energy flow), image sequence processing (motion coherence), machine learning feature engineering (enriched time-series representations), and other domains involving oscillatory or periodic data.

### BRIEF DESCRIPTION OF THE DRAWINGS

FIG. 1 is a flowchart illustrating an exemplary multi-stage process for extracting relational and harmonic features from signals, including cepstrum computation, marker derivation, phase-space resonance correlation, higher-order spectral analysis on time-series evolution, integral coherence score aggregation, causal chain map construction, and resource/priority identification.

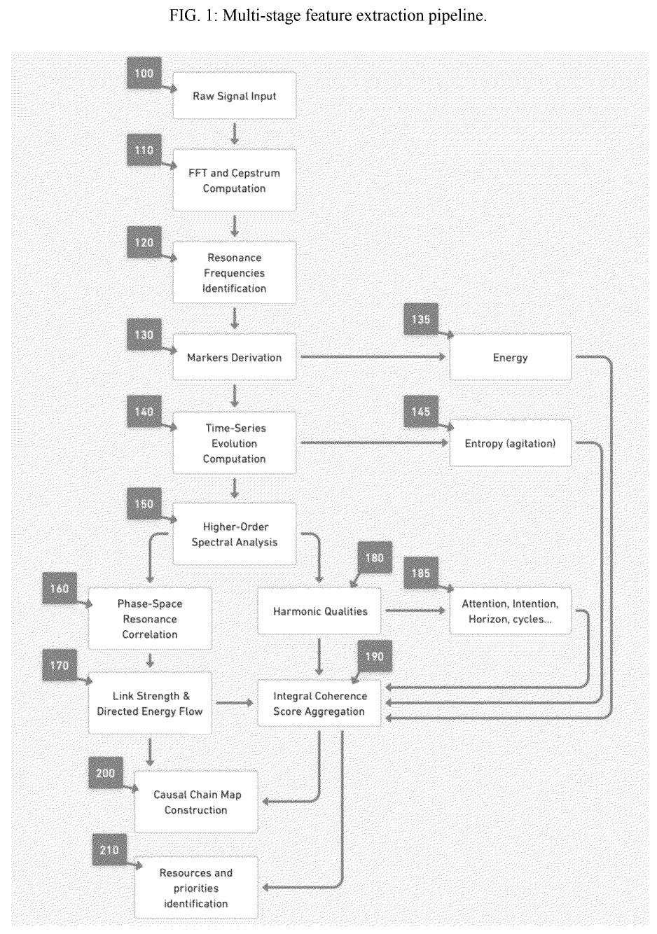

1. A computer-implemented method for extracting relational and harmonic features from time-series or multi-component signals, comprising:
computing cepstrum of the signal to identify main resonance frequencies; deriving multiple markers from the signal or sub-components; correlating common resonance frequencies across markers in phase space to compute link strength, treating markers as oscillators with momentum at resonance points; computing time-series evolution of each marker; applying higher-order spectral analysis on the evolution series to derive harmonic qualities including perceptual spread (attention), skewness (intention), and long-term trend (horizon); aggregating an integral coherence score per marker from energy, entropy, harmonic qualities, and link strengths.

2. The method of claim 1, wherein energy transfer directionality is determined by resonance links, with energy flowing from higher-energy markers to lower-energy markers through shared resonances.

3. A system comprising processor and memory executing the method of claim 1.

4. The method of claim 1, wherein link strength is weighted by cepstrum power at common resonance frequencies.

5. The method of claim 1, wherein energy transfer directionality is determined by resonance links, with energy flowing from higher-energy markers to lower-energy markers through shared resonances in phase space.

6. The method of claim 1, wherein causal chains are constructed as navigable maps of markers and successive resonance links, enabling analysis of energy/information flow paths.

7. The method of claim 1, wherein an integral coherence score is computed for each marker as a weighted combination of energy from cepstral/FFT bins, entropy (agitation level), harmonic qualities derived from higher-order spectral analysis, and relational link strengths to other markers.

8. The method of claim 7, wherein resources are identified as markers with high integral coherence score (high energy, low entropy, balanced harmonic qualities, strong outgoing links), and priorities as markers with low integral coherence score.

9. The method of claim 1, applied to physiological or biological signals such as electrocardiogram (ECG), electroencephalogram (EEG), photoplethysmogram (PPG), or any series reflecting biological or physiological data, to extract biologically meaningful biomarkers.

10. The method of claim 1, applied to vibration or machinery signals to extract relational fault propagation and harmonic stability features for predictive maintenance.

11. The method of claim 1, applied to audio or acoustic signals to identify harmonic relationships and inter-component influences in polyphonic or complex sound environments.

12. The method of claim 1, applied to radar or sonar returns to detect resonant objects or hidden correlations through phase-space linked resonances.

13. The method of claim 1, applied to seismic or geophysical time-series to model energy flow and coherence between wave components or subsurface layers.

14. The method of claim 1, applied to image sequences or video frames treated as time-series signals to extract relational motion coherence or dynamic texture features.

15. The method of claim 1, used as a feature extraction step in machine learning pipelines to generate enriched representations from time-series or multi-component input data for improved classification, anomaly detection, or forecasting.

16. The method of claim 1, wherein the extracted features and integral coherence scores are provided as auxiliary input or supervision to large language models, multimodal transformers, or other deep learning architectures to enhance reasoning, generation, or adaptation on sequential or oscillatory data.
